(19) United States
(12) Patent Application Publication
(10) Pub. No.: US 2003/0106165 A1
(43) Pub. Date: Jun. 12, 2003

(54) Method for highlighting and permanent waving in a single sitting

(76) Inventor: Sandra Guy Brown, Raleigh, NC (US)

Correspondence Address: Ms. Sandra G. Brown, 6104 Donnybrook Road, Raleigh, NC, 27606-9774, US

(21) Appl. No.: 10/007,541

(22) Filed: Dec. 10, 2001

(51) Int. Cl. A61K 7/13

(52) U.S. Cl. 8/405; 8/404

(57) ABSTRACT

A method for highlighting, or coloring selected portions of, and waving a person's hair in a single sitting. Only selected portions of the hair receive the waving treatment. In the same sitting, other selected portions of the person's hair receive a coloring treatment to attain, in those other selected portions, a color that is either lighter or darker than the subject's natural or preexisting hair color. The proportion of the subject's hair that receives the waving treatment and the proportion that receives the highlighting treatment may vary according to the subject's desires, but none of the subject's hair receives both the waving and the highlighting treatment.

### BACKGROUND OF THE INVENTION

It has long been fashionable for persons, women especially, to curl their hair by means of a process known as permanent waving. It has also long been fashionable for persons to use coloring agents to change the color of their hair to other than their natural hair color.

Many prior art methods have attempted to color and wave hair at the same time. In many of those prior art processes, the stylist applies the coloring and permanent waving agents at the same time. U.S. Pat. No. 4,781,724 (issued Nov. 1, 1988 to Wajaroff, et al.), for example, discloses a method for the simultaneous dying and permanent waving of hair. In the Wajaroff method, a coloring chemical composition is added to the permanent wave fixing chemical composition in order to prevent unwanted yellow or gray tones in the hair. Other prior art disclosures similar to Wajaroff include U.S. Pat. No. 5,188,639 (issued March 1992 to Schultz et al.) and U.S. Pat. No. 5,161,553 (issued November 1992 to Cohen et al.). Schultz discloses a method of mixing a coloring agent with a permanent waving agent, thus applying two chemical compounds to each strand of hair. Cohen discloses a method for waving and coloring hair in an essentially simultaneous process where the coloring agent is applied not with the permanent waving agent but with the post-waving neutralizing agent. All of these prior art methods envision waving and coloring all of a subject's hair at the same time and do not allow for highlighting or waving just a portion of the subject's hair.

Due to the characteristics of the permanent waving and coloring agents commonly used today as well as the nature and composition of human hair, both waving and coloring can result in damage to the hair. In prior art methods of simultaneous or near simultaneous waving and coloring, the hair suffers from the damaging effects of both the waving and coloring agents, compounding the total damage to the hair. To minimize the risk of damage to the hair, many stylists recommend that a subject receive a permanent waving treatment in one sitting and a coloring treatment in another, with the two sittings being spaced about two weeks apart. Since each treatment requires about two hours, the result of this practice is to increase the time spent and expense incurred by the subject desiring both wave and coloring treatment. And even with such an interval, however, the hair may still suffer damage from the two procedures.

Many hairdressing clients do not desire the overall curly head of hair that results from a full permanent wave treatment and instead, want a head of hair that is not so much curly as having more bounce and fullness In addition, many hairdressing clients may not desire to change the color of all the hair on their head but rather to add highlights. Highlights are small portions of a subject's hair that are either lighter or darker than the main hair coloring and highlighting is a technique in which coloring agents are used to change those selected portions to the desired or lighter or darker hair color.

What is needed is a method by which a hairdressing subject may achieve a hair style that has the desired bounce and fullness and with highlights while, at the same time, minimizes the risk of damage to the hair, time spent by the subject in the styling procedure and expense to the subject.

### SUMMARY OF THE INVENTION

The present invention is a method of selectively waving and selectively coloring, or highlighting, a subject's hair in the same sitting. Only selected portions of the subject's hair receive the permanent waving treatment while other selected portions of the hair receive the highlighting treatment. The proportion of a subject's hair that receives the waving treatment and the proportion that receives the highlighting treatment may vary according to the effect or effects that the subject and the stylist desire to achieve but no portion of a subject's hair receives both a waving treatment and a highlighting treatment in the same sitting. Since any given portion of a subject's hair receives only the waving treatment or the coloring treatment in the same sitting, the subject's hair is not subjected to the undesirable effects that result from a combined waving and coloring process. The risk of damage from the two treatments is therefore minimized.

The stylist may perform a number of ancillary processes or procedures, such as washing and drying the subject's hair, as necessary supplements to the fundamental steps of waving and highlighting.

The method of the present invention envisions using materials and techniques that are well-known in the art to perform the waving portion of the method and, as well, using well-known materials and techniques to perform the highlighting portion of the method. It is the combination of the two conventional processes in a novel way that is the essence of the method of the present invention.

### DESCRIPTION OF THE DRAWINGS

FIG. 1 is a partial view of a subject's head and hair while a coloring agent is applied to a selected portion of the hair.

FIG. 2 is a partial view of a subject's head and hair while wrapped during the coloring process.

FIG. 3 is a partial view of a subject's hair while a selected portion is rolled for permanent waving.

### DESCRIPTION OF THE PREFERRED EMBODIMENT

This description of the preferred embodiment of the present invention is couched in terms of a stylist practicing the method of the invention on the hair of a subject. This is the normal way in which the method would be used. It is feasible that a subject could practice the method on her own hair but such would be awkward and time-consuming.

As a first step in the highlighting and waving operation, the stylist normally washes and towel dries the subject's hair. This step is not required but desirable, for damp hair is more manageable than dry.

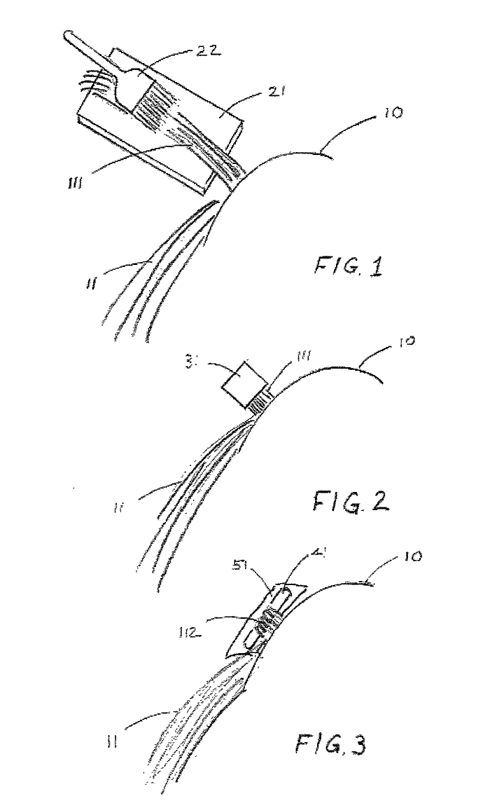

The stylist then selects portions of the subject's hair for highlighting. The stylist applies a coloring agent, such as bleach or a dye, to a portion so selected as shown in FIG. 1. FIG. 1 shows a selected portion of hair 111 separated from remainder of the hair 11 on the subject's head 10. The stylist lays out selected portion of hair 111 on pallet 21 and applies the coloring agent to portion 111 using brush 22. The stylist then wraps portion 111 in wrapper 31, which is made from a suitable liquid-impermeable material such as aluminum foil, to keep the coloring agent in contact with the selected portion and to prevent the coloring agent from coming in contact with other portions the subject's hair 11. The stylist then repeats this step on other selected portions of hair that are intended to be highlighted.

The stylist then selects other portions of the subject's hair for waving. The stylist may first apply a solution to a portion so selected and may place a strip of absorbent material 51, made from a material such as cotton, under the selected section. Then, as shown in FIG. 3, the stylist rolls a selected portion of hair 112 on rod 41. After rolling, the stylist applies a waving agent to portion 112. The stylist then repeats this step on other selected portions of hair that are intended to be waved.

After completing the application of the coloring and coloring agents to selected portions, the subject's head then has numerous wrapped highlighted portions and numerous rolled-for-waving portions. There may also be some portions of hair that have not received either a highlighting treatment or a waving treatment The fractions of the subject's total head of hair that receive highlighting, waving or no treatment can vary depending on the desired effect to be attained. The highlighted portion may vary from 25 to 75 percent of the subject's hair and the waved portion may also vary from 25 to 75 percent. But in no case does a selected portion receive both a highlighting and a waving treatment The stylist then covers the subject's hair with a cap and, if a particular coloring or waving agent requires it, applies heat to the hair.

The coloring and waving agents normally used require that the agents remain in contact with the hair being colored or waved for some predetermined time After the passage of that time, the stylist removes the wrapping from the portions selected for highlighting and then rinses the coloring and waving agents from the subject's hair with water The stylist then towel dries the hair.

The stylist then applies a neutralizer to the hair portions rolled on the rods and allows the neutralizer to remain on those portions for the required time. After the passage of that time, the stylist rinses the neutralizer from the hair with water.

Finally, the stylist towel dries the subject's hair and; styles it as usual.

I claim:

1. A method for coloring portions of hair on a person's head while at the same sitting waving other portions of said hair comprising the steps of
selecting first portions of said person's hair for coloring;
coloring said selected first portions;
selecting second portions of said person's hair for waving and
waving said second portions.

2. A method for highlighting portions of hair on a person's head while at the same sitting waving other portions of said hair comprising the steps of
selecting first portions of said person's hair for highlighting;
applying a coloring agent to said selected first portions;
sealing said coloring agent with said selected first portions to isolate said first portions from other portions of said person's hair as well as said person's scalp;
selecting second portions of said person's hair for waving;
applying a pre-wrap solution to said second portions;
applying means to isolate said second portions from said person's scalp;
wrapping strands of hair from said second portions on rods;
applying a waving agent to said strands wrapped on said rods;
covering said person's hair;
waiting with said coloring agent applied to said first hair portions and with said waving agent applied to said second hair portions;
removing said isolation means;
removing said coloring agent from said first hair portions and said waving agent from said second hair portions;
applying a neutralizing agent to said second hair portions;
waiting with said neutralizing agent applied; and
removing said neutralizing agent from said second hair portions.

3. The method of claim 2 further comprising the additional step of washing said person's hair, said additional step to be performed before performing said “selecting first portions” step of claim 2.

4. The method of claim 2 in which said “removing said isolation means” and said “removing said coloring dye and said waving agent” steps are performed simultaneously.

5. The method of claim 2 in which said “removing said coloring dye and waving agent” and said “removing said neutralizing agent” steps are accomplished by rinsing with water.

6. The method of claim 2 further comprising the additional step, to be performed twice, of
drying all of said person's hair; said first additional “drying” step to be performed after said “removing said coloring agent” step and before said “removing said neutralizing agent step, and said second additional “drying” step to be performed after said “removing said neutralizing agent” step.

7. The method of claim 6 in which both said “drying all of said person's hair” steps are accomplished by drying with a towel.

8. The method of claim 2 in which said “waiting” in each of said “waiting” steps is for a predetermined time.
